(19) United States
(12) Patent Application Publication
(10) Pub. No.: US 2009/0215693 A1
(43) Pub. Date: Aug. 27, 2009

(54) METHODS FOR INCREASING HYALURONIC ACID LEVELS AND IMPROVING MOISTURE RETENTION IN TISSUE

(76) Inventors: Loren S. Ward, Twin Falls, ID (US); Kevin Thomson, Fitchburg, WI (US)

Correspondence Address: DONNA J. RUSSELL, 1492 ANTHONY WAY, MT. JULIET, TN, 37122, US

(21) Appl. No.: 12/358,209

(22) Filed: Jan. 22, 2009

Related U.S. Application Data
(60) Provisional application No. 61/022,726, filed on Jan. 22, 2008.
(60) Provisional application No. 61/024,265, filed on Jan. 29, 2008.

(51) Int. Cl. A61K 38/16

(52) U.S. Cl. 514/12

(57) ABSTRACT

Disclosed is a method for increasing hyaluronic acid levels in tissue and increasing moisture retention in skin.

### CROSS-REFERENCE TO RELATED APPLICATIONS

This application claims the benefit of priority of earlier-filed U.S. Provisional Patent Applications No. 61/022,726 (filed Jan. 22, 2008) and 61/024,265 (filed Jan. 29, 2008).

### FIELD OF THE INVENTION

The present invention relates to methods for increasing levels of factors secreted by connective tissue cells. More specifically, the invention relates to compositions and methods for their use in increasing the levels of hyaluronic acid in mammalian tissue.

### BACKGROUND OF THE INVENTION

Fibroblasts are connective tissue cells that provide a structural framework for many tissues by secreting precursors of the extracellular matrix. Fibroblasts make and secrete collagen proteins and are involved in wound healing and scar formation. They may be found just beneath the surface of the skin, and provide part of the support structure for tissues and organs.

Among the factors that are secreted by fibroblasts is the polysaccharide hyaluronic acid (HA, also known as sodium hyaluronate or hyaluronan), a jelly-like substance that fills the space between collagen and elastin fibers. HA occurs naturally in the body and is found in all mammals. The highly-conserved structure of HA comprises linear, unbranched polyanionic disaccharide units consisting of glucuronic acid and N-acetyl glucosamine, joined alternately by beta 1-3 and beta 1-4 glucosidic bonds. The glycosaminoglycan family to which it belongs also includes chondroitin sulphate and heparan sulphate.

HA is viscoelastic, forms hydrogen bonds with water, and has a significant capacity for binding and retaining water, especially if the HA is a higher molecular weight species. It is therefore important for tissue hydration and lubrication. Higher amounts of HA are found in the skin, vitreous body and connective tissues. In the synovial fluid of the joints, it acts as a “shock absorber,” and its loss during the aging process is often associated with pain and dysfunction of those joints.

Hyaluronic acid acts as a moisture retention agent for the skin. A number of HA products, primarily gels, are commercially available for use as dermal fillers to improve the skin's contour and reduce depression that have been formed as the result of scars, injury, or lines and wrinkles associated with aging. Furthermore, HA is associated with transport of essential nutrients from the bloodstream to the skin cells.

When used as dermal filler, HA is often applied by injecting small amounts into the skin. These treatments are generally repeated at intervals in order to maintain the desired effects (e.g., diminishing lines and wrinkles, firming skin). HA topicals may provide benefits such as moisturizing the skin, providing a barrier to moisture loss, and increasing the elasticity of the skin.

HA has traditionally been extracted from rooster combs and bovine vitreous humor. Because it is difficult to isolate higher molecular weight HA economically from these sources and the demand for HA continues to increase, bacterial fermentation systems have more recently been used for HA production. Streptococcal fermentations have been used because Streptococcus species produce HA for capsule formation, but these systems generally produce HA with lower molecular weight. Furthermore, the yield of HA from these bacterial systems has generally been too low to meet the increasing market demand.

It would be desirable to identify agents that induce in vivo hyaluronic acid synthesis, both from the perspective of the efficacy of the molecule produced and the ability to achieve delivery without significant degradation of HA. A number of conditions are improved by the administration of HA, so the demand for HA and the need to stimulate its production within the body may be expected to increase over the coming years.

### SUMMARY OF THE INVENTION

The invention relates to methods for using therapeutically effective amounts of lactoferrin for increasing hyaluronic acid levels in mammalian tissue. In one aspect, such a method comprises applying to the skin of a mammal a therapeutically-effective amount of lactoferrin to increase hyaluronic acid levels in the skin tissue. In another aspect, the method comprises applying to the skin of a mammal a therapeutically-effective amount of lactoferrin to increase moisture retention in the skin.

Compositions for use in the method of the invention comprise, consist essentially of, and/or consist of compositions having a lactoferrin content of at least about 0.1% by weight, but may also comprise at least about 0.2%, 0.3%, 0.4%, 0.5%, 1.0%, 5.0%, etc., lactoferrin. Compositions for use in the method of the invention may also incorporate as ingredients mammalian milk fractions comprising lactoferrin in an amount to provide sufficient lactoferrin for therapeutic efficacy. In one aspect, such a milk fraction may have at least about two percent (2%) lactoferrin by weight. In some aspects of the invention, such mammalian milk fractions may be incorporated into one or more cosmetic compositions for topical application to the skin, to provide cosmetic compositions comprising at least about 0.1%, and more preferably at least about 0.2%, at least about 0.3%, at least about 0.4%, at least about 0.5%, etc., lactoferrin by weight.

### BRIEF DESCRIPTION OF THE DRAWINGS

FIG. 1 is a graph of the results shown in Table 1 herein. Hyaluronic acid levels are expressed as ng/ml in fibroblasts treated with the indicated bovine milk-derived products as described in the Examples. (S—sodium butyrate, U—untreated, D—DBcAMP.) All products were produced by Glanbia Nutritionals, Inc., Twin Falls, Id.

### DETAILED DESCRIPTION

The inventors have discovered that application of lactoferrin to fibroblast cells increases hyaluronic acid levels in fibroblast tissue. The inventors have also determined that incorporation of lactoferrin, which may be in the form of a milk-derived product containing lactoferrin, into a topical cosmetic product significantly increases the moisture content of human skin when the lactoferrin concentration in the topical cosmetic product is at least about 0.1%, and more preferably at least about 0.2%, at least about 0.3%, at least about 0.4%, at least about 0.5%, at least about 1%, at least about 5%, etc., by weight.

Where the terms “comprise” or “comprises” are used herein, the inventors intend that the transitional phrases “consist(s) essentially of” and/or “consist(s) of” may also apply. Furthermore, two forms of lactoferrin may be used interchangeably in the method of the invention: an isolated lactoferrin (e.g., an isolated lactoferrin protein, a functional subunit of a LF protein, and/or a peptide derived from the sequence of a mammalian LF) and a lactoferrin-enhanced milk-derived product (e.g., a milk fraction having a lactoferrin content that is higher than that of the milk from which the fraction is isolated and/or separated, and preferably a whey protein product having a lactoferrin concentration of at least about 2%, and more preferably at least about 5%, by weight). It is also to be understood that suggested ranges may include sub-ranges therein (e.g., at least about 3% to about 99%, at last about 5% to about 98%, at least about 10% to about 100%, at least about 15% to about 100%, at least about 15% to about 90%, etc.). For simplicity, the term “lactoferrin” (LF) will be used herein to identify isolated lactoferrin, the term “isolated” intended to mean that steps have been taken to purify lactoferrin from its original source and to isolate it from other compounds with which it may have been associated. Likewise, the term “lactoferrin-enhanced milk-derived product” (LEMP) will be used herein to identify milk fractions for which additional processing steps have been taken that result in increasing the lactoferrin content of the product to a level greater than that of the milk from which it is derived, and more preferably to a level of at least about 2% by weight to about 100% by weight, and sub-ranges within this range.

Lactoferrin is a multi-functional protein found in mammalian milk, primarily in the whey fraction. Commercially-available milk-derived products include whey protein concentrates (WPCs) and whey protein isolates (WPIs). WPCs are produced by physical separation techniques to remove sufficient non-protein components to result in the production of a finished dry product that contains not less than 25 percent protein. It may be supplied and used as a fluid, concentrate, or dry product. Protein ranges for WPC generally fall into the 34-80% range, and include beta-lactoglobulin, alpha-lactalbumin, glycomacropeptide (if derived from sweet whey) as major protein components, with lactoferrin, lactoperoxidase, immunoglobulins and other proteins comprising a minor fraction. Current WPC products generally contain less than 1 gram lactoferrin per 100 grams WPC, although some may contain up to 2% lactoferrin. WPI is similarly obtained, with the distinction that it has a lower fat content (<1% as compared to 3-6% for WPC). The lactoferrin content of WPI is generally about 0.5-1% by weight. LEMP for use in the method of the present invention may even more preferably be produced by methods known to those of skill in the art to concentrate and/or isolate milk proteins to provide products having at least about two percent (2%) to 100% lactoferrin by weight, with a lactoferrin concentration of at least about five percent (5%) being preferred in order to maximize the effect of the lactoferrin on hyaluronic acid production and moisture retention in the skin. Methods for concentrating or isolating the lactoferrin protein in or from a milk-derived product are known to those of skill in the art.

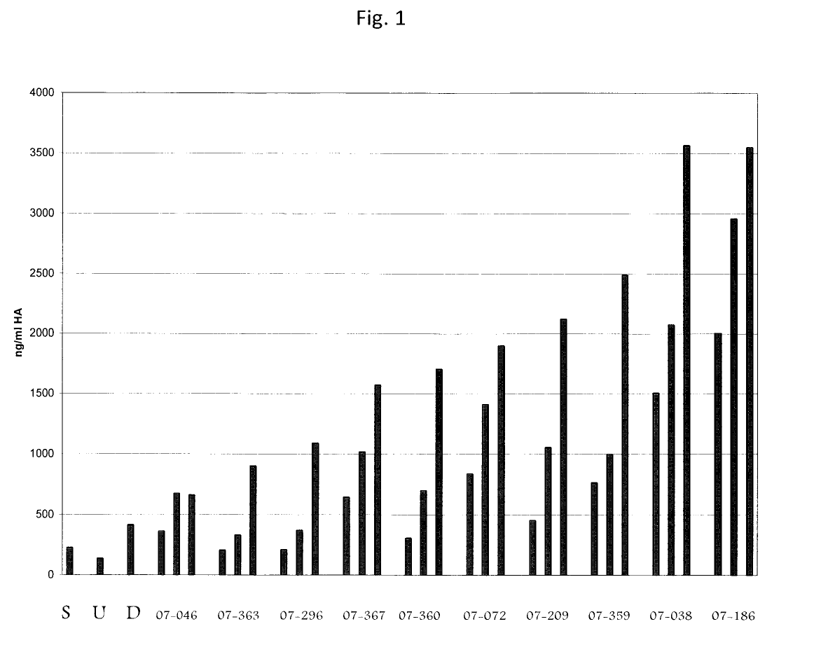

When compared with hyaluronic acid levels from untreated fibroblasts, a detectable and significant increase in hyaluronic acid levels was noted in a fibroblast culture treated with bovine milk-derived lactoferrin, even at the lowest concentrations tested. Furthermore, dibutyryl cyclic AMP (DBcAMP) is known to significantly increase hyaluronic acid production by cells, but bovine-derived LEMP products provided an increase in fibroblast-derived hyaluronic acid levels over that provided by treatment of cells with DBcAMP. (See FIG. 1 and Table 1.) Two products used in these tests, 07-186 and 07-038, were products comprising 97% protein on a dry basis, with greater than 95% of total protein being lactoferrin. The iron content of 07-038 was less than 15 mg/100 grams of protein, and the moisture content was 5%.

The inventors have demonstrated that LF and LEMP significantly increase fibroblast-produced hyaluronic acid levels. The invention therefore provides a method for using LF and/or LEMP for increasing hyaluronic acid in tissues for dermatological, rheumatological, opthalmological, surgical, wound healing (e.g., decreasing scar formation), and other uses where it is desirable to have increased levels of hyaluronic acid in the tissues. The LF may be of any mammalian origin, including, for example, bovine and/or human, and may also be a subunit of a LF protein, such as a peptide derived from the sequence of a mammalian LF. It is also within the scope of the invention to utilize LF or a peptide therefrom that is produced via a recombinant system. Peptides having at least a portion of the LF protein amino acid sequence and the functional capacity to increase hyaluronic acid in tissue are within the scope of the invention, as are proteins comprising the sequence of a lactoferrin protein with or without additional amino acids at the amino terminal and/or carboxy terminal end(s). Amino acid substitutions may also be made within the protein or peptide sequence, provided that those amino acid substitutions conserve the functionality of the protein or peptide. The LEMP generally may also be from any mammal. Bovine LEMPs may be especially useful, as they provide a significant source of LF necessary to meet the demand for use in preparations for topical, injectable, and other uses.

In one embodiment, a method is provided for increasing hyaluronic acid in the skin. Increasing hyaluronic acid levels in skin tissue can increase tissue hydration, improve the contour of the skin, reduce wrinkles and fine lines, reduce scar formation, and reduce the appearance of existing scars. In studies done with female volunteers, a topical composition comprising approximately 1% lactoferrin (with LEMP used as the lactoferrin source) increased the moisture content of the skin approximately 7.50% after 1 week, 10.15% after 2 weeks, and 16.81% after 4 weeks. Topical compositions for use in increasing tissue hydration may comprise substantially purified lactoferrin protein, fragments thereof, and/or lactoferrin peptides. Lactoferrin-enhanced milk-derived products having a concentration of at least about 2% lactoferrin by weight may also be used to produce topical compositions. Such LEMPs may more preferably contain at least about 5% lactoferrin. One LEMP that the inventors have demonstrated to be effective for use in compositions for increasing hyaluronic acid levels and in increasing tissue hydration is Bioferrin®, produced by Glanbia Nutritionals, Inc., Monroe, Wis. Bioferrin® has a protein concentration of greater than 97% on a dry basis, with greater than 95% of that protein being bovine lactoferrin.

Compositions for topical application may be effective for significantly increasing tissue hydration when lactoferrin levels are at least about 0.1% in the topical composition to be applied to the skin, but the inventors suggest that at least about 0.5% and more preferably at least about 1%, for example, may be used to further improve hydration and increase visible effects. Topical compositions for cosmetic use generally comprise additional ingredients such as, for example, carriers, excipients, moisturizing agents, water, antimicrobial agents, and colorings. LF may be admixed into such a topical composition to provide a final concentration of LF of at least about 0.1%. A LEMP such as Bioferrin® may also be admixed into a composition for topical application, so that the composition provides a final concentration of at least about 0.1% LF, and more preferably at least about 0.5% LF, at least about 1% LF, etc.

Hyaluronic acid accelerates wound healing and reduces scar formation. LF and/or LEMPs may be used in the method of the invention to increase hyaluronic acid levels in skin tissue to decrease scar formation during wound healing. This may be especially beneficial for surgical incisions, cuts, scrapes, and other injuries to skin located on the arms, legs, face, and neck. Taken orally as a post-operative therapy or applied as a sterile composition to a surgical site prior to wound closure, it may improve wound healing and decrease scar formation in and around surgical sites involving internal organs which may not be accessible for direct application to the tissue.

LEMPs provide easy-to-administer compositions (via oral, topical, or other means of administration) for increasing hyaluronic acid levels. Lactoferrin derived from bovine milk is safe for human consumption and topical application, as are bovine milk-derived products, as has been shown for both WPIs and WPCs. Use of lactoferrin for increasing hyaluronic acid levels provides a very desirable solution to the problems posed by production and administration of hyaluronic acid, such as molecular weight differences between hyaluronic acid molecules produced in different systems, enzymatic degradation of exogenous hyaluronic acid, and difficulty of use of hyaluronic acid for cosmetic and topical formulations. Hyaluronic acid's affinity for water presents a challenge for formulating a variety of products in which it might be incorporated. LF, on the other hand, is much more easily incorporated into a composition and can increase the natural levels of HA in the skin itself.

For topical application, LF may be provided in gels, oils, lotions, creams, and a variety of other cosmetically-suitable carrier preparations. Such preparations may also comprise additional ingredients having beneficial effects on the skin (e.g., vitamin E). Topical preparations may also comprise agents for increasing absorption of active agents, such as pluronic lecithin organogels. Topical preparations may also comprise carriers, antibacterial agents, preservatives, coloring agents, and other agents and ingredients known to those of skill in the art of cosmetic formulation. Waxes and other suitable ingredients may be used where application to skin such as the skin of the lips may be desired. Topical compositions comprising lipsticks, lip glosses, lip balms, and other preparations appropriate for application to the tissue of the lip may provide effective application of LF and/or LEMP at levels to provide a topical lip formulation comprising at least about 0.1%, and more preferably at least about 1.0% lactoferrin by weight, to promote hydration and moisture retention of the tissue.

Hyaluronic acid has been used experimentally in conjunction with bone graft materials to increase incorporation of the bone graft material. Bone graft materials are known to those of skill in the art, and certain of those materials may be combined with, complexed with, or contain active agents such as hyaluronic acid. Bone grafts having depots for bioactive agents for promoting incorporation of the graft have been described. One such bone graft design is disclosed in U.S. 20060293757A1 (McKay, et al.) LF and/or LEMP may be coated on the surface of a bone graft material, may be incorporated into the bone graft material, or may be placed in one or more depots within a bone graft to promote the increase of HA levels in the adjacent tissue. Therefore, the invention provides a method for increasing HA levels in the tissue adjacent to a bone graft by providing the bone graft in combination with a therapeutically-effective amount of LF incorporated into the bone graft or deposited at the surface of the bone graft to provide increased levels of tissue HA in the area surrounding the bone graft. Lactoferrin may also be provided in the area near the bone graft by means of modified-release pharmaceutical carriers or depots, matrices, or other devices or compositions that may release lactoferrin within the tissue space surrounding the graft.

Hyaluronic acid also increases the tensile strength of the ligaments that secure teeth in place. The present invention also provides a method for improving tooth and gum health by administering LF and/or LEMP to increase HA levels in the tissue surrounding a mammalian tooth. In humans, this may be easily provided for oral administration via a chewing gum, coated strip, dissolvable gel, or other product that may provide a therapeutically effective concentration of LF to the tooth/gum junction. For example, LF and/or LEMP may be provided in a modified-release, dissolving strip (e.g., a pullulan matrix) that may be placed along the gum line or may be provided in combination with a non-dissolving strip that may be placed over the teeth and inner and outer gum lines to hold the lactoferrin and/or LEMP in place for a sufficient period of time to allow the lactoferrin and/or LEMP active agent to penetrate the gum line to promote hyaluronic acid production.

Because it is readily available as well as effective, bovine LF from LEMP provides an especially available source of LF for purposes such as topical application for the treatment of the skin or for oral application for improving or maintaining tooth/gum attachment. For bone graft applications, it may be more desirable in some circumstances to utilize human LF.

Therapeutic levels of LF for use in the method of the invention may be determined by those of skill in the art of pharmaceutical, nutriceutical, cosmetic, or food formulation, depending upon the specific use to which the method will be applied, based upon the knowledge of those of skill in the art in these fields and the information provided herein by means of the Examples provided. The method of the invention may be practiced by the application of topical lotions, creams, serums, etc. to the skin. It may also be practiced by the application of shampoos containing LF and/or LEMP to increase moisture retention in scalp tissue. It may be practiced by the application of topical lotions, creams, serums, and/or other carriers to the skin of individuals having medical conditions characterized by the presence of very dry skin. For the treatment of skin conditions characterized by excessive dryness, it may be preferable to incorporate LF and/or LEMP into a topical composition at a level of at least about 3%, and more preferably at least about 10%, by weight.

The method of the invention may be useful for human and/or animal use. For veterinary use, topical compositions may be prepared so that at least about 0.1%, and more preferably at least about 1% lactoferrin, for example, may be applied to the skin of a dog, cat, or other animal to increase moisture retention in the skin tissue. Shampoos, and especially rinses, containing at least about 0.1% lactoferrin may provide effective carriers for the application of lactoferrin to the skin of a cat, dog, horse, or other animal.

The invention may be further described by means of the following non-limiting examples.

### Example 1

Fibroblasts were seeded into individual wells of a 12-well plate in 1.0 ml of Fibroblast Growth Media (FGM) and incubated overnight at 37±2° C. and 5±1% CO₂. On the following day the media was removed via aspiration to eliminate non-adherent cells and replaced with 1.0 ml of fresh FGM. Cells were grown until confluent, with a media change every 48 to 72 hours. Upon reaching confluency the cells were treated for 24 hours with DMEM supplemented with 1.5% FBS to eliminate growth factors from the normal culture media. After this 24-hour wash-out period the cells were treated with the indicated test materials (i.e., milk-derived products containing lactoferrin) at the specified concentrations dissolved in FGM with 1.5% FBS. Dibutyrl cAMP (DBcAMP) at 0.1 mM was used as a positive control. Untreated cells (negative control) received FGM with 1.5% FBS. The cells were incubated for 48 hours and at the end of the incubation period cell culture medium was collected and either stored frozen (−75° C.) or assayed immediately. All testing was done in triplicate.

A series of hyaluronic acid standards from 50 ng/ml to 3,200 ng/ml was prepared. Next, 100 μl of each standard (in duplicate) and sample was transferred to a well in an incubation plate. After adding 50 μl of detection solution to each well (except the reagent blank wells) the plate was incubated for 1±0.25 hour at 37±2° C. After incubation, 100 μl of each sample/standard from the incubation plate was transferred to a corresponding well in an ELISA plate. The ELISA plate was covered and incubated for 30±5 at 4° C. and then washed three times (3×) with 300 μl of wash buffer. After the final wash, 100 μl of enzyme solution was added to each well and the plate was incubated at 37±2° C. for 30±5 minutes. After this incubation, the wells were washed again as described above and 100 μl of enzyme substrate solution was added to each well. The plate was incubated for 30-45 minutes at room temperature. After incubation, 50 μl of stop solution was added to each well and the absorbance was measured at 405 nm using a plate reader. Hyaluronic acid production was expressed as ng/ml and results are shown in Table 1 and the graph of FIG. 1.

Some of the tested products contained no measurable amounts of native (non-denatured, non-hydrolyzed) lactoferrin (07-046, 07-296, for example). Product 07-359 was a whey protein concentrate modified to selectively concentrate certain proteins (<2% native lactoferrin), 07-360 was a whey protein isolate (≦0.5% native lactoferrin), and 07-038 and 07-186 were milk protein isolates having approximately 97% protein, with lactoferrin comprising approximately 95% of the total protein.

TABLE 1

| Agent Applied | Amount Applied | HA Measured |
|---|---|---|
| 07-038 | 1% | 3549 ± 177* |
| 07-038 | 0.1% | 2960 ± 108* |
| 07-038 | 0.01% | 2005 ± 165* |
| 07-046 | 1% | 663 ± 108* |
| 07-046 | 0.1% | 676 ± 59* |
| 07-046 | 0.01% | 365 ± 10 |
| 07-072 | 1% | 1900 ± 52* |
| 07-072 | 0.1% | 1413 ± 150* |
| 07-072 | 0.01% | 838 ± 60* |
| 07-186 | 1% | 3566 ± 158* |
| 07-186 | 0.1% | 2073 ± 284* |
| 07-186 | 0.01% | 1507 ± 119* |
| 07-209 | 1% | 2122 ± 182 |
| 07-209 | 0.1% | 1056 ± 91* |
| 07-209 | 0.01% | 455 ± 58 |
| 07-296 | 1% | 1091 ± 125* |
| 07-296 | 0.1% | 374 ± 30 |
| 07-296 | 0.01% | 212 ± 20 |
| 07-359 | 1% | 2491 ± 476* |
| 07-359 | 0.1% | 1001 ± 23* |
| 07-359 | 0.01% | 766 ± 42* |
| 07-360 | 1% | 1705 ± 53* |
| 07-360 | 0.1% | 700 ± 90* |
| 07-360 | 0.01% | 308 ± 43 |
| 07-363 | 1% | 904 ± 96* |
| 07-363 | 0.1% | 333 ± 29 |
| 07-363 | 0.01% | 208 ± 12 |
| 07-367 | 1% | 1572 ± 129* |
| 07-367 | 0.1% | 1018 ± 139* |
| 07-367 | 0.01% | 644 ± 78* |
| Sodium Butyrate | 4 mM | 139 ± 6 |
| DBcAMP | 0.1 mM | 416 ± 24 |
| Untreated | | 227 ± 5 |

*denotes values that are significantly different from the Untreated group (p < 0.05).

### Example 2

Thirty females between the ages of 40 and 60 who had previously used moisturizer at least once a week were recruited for the study. All study participants were asked to refrain from using moisturizers for at least two days prior to onset of treatment, as well as to abstain from shaving the test site at least 48 hours prior to and during the test period.

The inner forearm, midway between the wrist and the elbow, was designated as the test area. One forearm served as the untreated control, while the other forearm received treatment with the test material, designated according to a randomized complete block design. Test panelists were asked to apply the test product to the designated test site two times daily, ad-libitum for the study period. Participants were also instructed not to apply the test product for at least one hour prior to the measurements being taken.

On evaluation days, panelists were allowed to equilibrate to the ambient environment for 30 minutes prior to measurements being taken. Biophysical measurements consisted of electroconductivity-Novameter readings. Measurements were collected prior to the first application and again at 1 week, 2 weeks, and 4 weeks after commencement of the study. The test spot (1 inch by 1 inch) was located on the test site, 3 inches from the wrist.

Participants were instructed to keep a daily log noting the date, time and volume (compared to coin size) of each application, together with any subjective comments relating to product use. All samples were weighed prior to and after the study completion and the average amount of test material used per subject throughout the study period was calculated.

Electroconductivity was measured using a Nova Dermal Phase Meter, Model DPM 9003 (Nova, Technology Corp., Gloucester, Mass.), which measures skin surface impedance, providing a relative measure of the retained water content of the skin as a function of the skin's dielectric value. Skin impedance was recorded automatically when equilibrium was achieved. Novameter readings demonstrated that the test material increased the skin moisture content 7.50%, 10.15%, and 16.81% after 1, 2, and 4 weeks, respectively. All increases are considered statistically significant.

What is claimed is:

1. A method comprising applying a therapeutically effective amount of lactoferrin to a mammalian tissue to increase the level of hyaluronic acid in the tissue.

2. The method of claim 1 wherein the mammalian tissue comprises mammalian skin.

3. The method of claim 1 wherein the lactoferrin is provided by a lactoferrin-enhanced milk-derived product.

4. The method of claim 1 wherein the step of applying is performed by topical administration.

5. The method of claim 1 wherein the mammalian skin is human skin.

6. The method of claim 1 wherein the lactoferrin is bovine lactoferrin.

7. The method of claim 1 wherein the therapeutically effective amount of lactoferrin is an amount that is at least about 0.1% of a topical composition applied to the skin.

8. The method of claim 1 wherein the therapeutically effective amount of lactoferrin is an amount that is at least about 0.2% of a topical composition applied to the skin.

9. The method of claim 1 wherein the therapeutically effective amount of lactoferrin is an amount that is at least about 0.5% of a topical composition applied to the skin.

10. The method of claim 1 wherein the therapeutically effective amount of lactoferrin is an amount that is at least about 1% of a topical composition applied to the skin.

11. A method comprising applying to mammalian skin a therapeutically effective amount of lactoferrin to increase moisture retention in the skin.

12. The method of claim 11 wherein the step of applying is performed by topical application.

13. The method of claim 11 wherein the lactoferrin is from a lactoferrin-enhanced milk-derived product.

14. The method of claim 13 wherein the lactoferrin-enhanced milk-derived product is a bovine milk-derived product.

15. A method for decreasing scar formation in human skin, the method comprising administering to the skin a therapeutically effective amount of lactoferrin to increase hyaluronic acid and moisture retention in the skin.

16. The method of claim 15 wherein the lactoferrin is provided by incorporating a lactoferrin-enhanced milk-derived product into a topical composition.

17. The method of claim 17 wherein the milk-derived product is a bovine milk-derived product.
